(19) United States
(12) Patent Application Publication
(10) Pub. No.: US 2005/0245923 A1
(43) Pub. Date: Nov. 3, 2005

(54) Biopolar virtual electrode for transurethral needle ablation

(75) Inventors: Mark A. Christopherson, Shoreview, MN (US); Martin T. Gerber, Maple Grove, MN (US); Julie M. Woessner, St. Paul, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/835,193

(22) Filed: Apr. 29, 2004

(51) Int. Cl. A61B 18/14

(52) U.S. Cl. 606/41; 606/50

(57) ABSTRACT

A device and method for transurethral needle ablation of prostate tissue to alleviate BPH uses bipolar ablation needles and a virtual electrode. To create the virtual electrode, a conductive fluid is delivered to the target site within the prostate tissue. Ablation energy is then delivered to the target tissue and the virtual electrode via a pair of bipolar ablation needles. The ablation energy flows between the bipolar ablation needles, throughout the virtual electrode and the prostate tissue to create ablation lesions within the prostate.

### FIELD OF THE INVENTION

The invention relates generally to prostate treatment and, more particularly, to techniques for transurethral treatment of benign prostatic hypertrophy (BPH).

### BACKGROUND

Benign prostatic hypertrophy or hyperplasia (BPH) is one of the most common medical problems experienced by men over 50 years old. Urinary tract obstruction due to prostatic hyperplasia has been recognized since the earliest days of medicine. Hyperplastic enlargement of the prostate gland often leads to compression of the urethra, resulting in obstruction of the urinary tract and the subsequent development of symptoms including frequent urination, decrease in urinary flow, nocturia, pain, discomfort, and dribbling.

One surgical procedure for treating BPH is transurethral needle ablation (TUNA). The TUNA technique involves transurethral delivery of an electrically conductive needle to the prostate site. The needle penetrates the prostate in a direction generally perpendicular to the urethral wall, and delivers electrical current to ablate prostate tissue. The electrical current heats tissue surrounding the needle tip to destroy prostate cells, and thereby create a lesion within the prostate gland. The destroyed cells may be absorbed by the body, infiltrated with scar tissue or become non-functional.

U.S. Pat. No. 6,090,105 to Zepeda et al. discloses a multiple electrode ablation apparatus and method. U.S. Pat. No. 6,409,722 to Hoey et al. discloses an apparatus and method for creating, maintaining, and controlling a virtual electrode used for the ablation of tissue. U.S. Pat. No. 6,471,698 to Edwards et al. discloses a multiple electrode ablation apparatus. U.S. Pat. No. 6,537,272 to Christopherson et al. discloses an apparatus and method for creating, maintaining, and controlling a virtual electrode used for the ablation of tissue. U.S. Pat. No. 6,706,039 to Mulier et al. discloses a method and apparatus for creating a bipolar, virtual electrode for ablation of tissue. Leveillee, Raymond J., and Hoey, Michael F., “Radiofrequency Interstitial Tissue Ablation: Wet Electrode”, Journal of Endourology, Volume 17, Number 8, October 2003, discusses radiofrequency thermal therapy as delivered by a saline-augmented (“wet” or virtual) electrode. Table 1 below lists documents that disclose devices for transurethral ablation of prostate tissue.

TABLE 1

| Patent Number | Inventors | Title |
|---|---|---|
| 6,090,105 | Zepeda et al. | Multiple electrode ablation |
| | | apparatus and method |
| 6,409,722 | Hoey et al. | Apparatus and method for creat- |
| | | ing, maintaining, and controlling |
| | | a virtual electrode used for the |
| | | ablation of tissue |
| 6,471,698 | Edwards et al. | Multiple electrode ablation |
| | | apparatus |
| 6,537,272 | Christopherson et al. | Apparatus and method for creat- |
| | | ing, maintaining, and controlling |
| | | a virtual electrode used for the |
| | | ablation of tissue |
| 6,706,039 | Mulier et al. | Method and apparatus for creat- |
| | | ing a bipolar virtual electrode |
| | | used for the ablation of tissue |
| Publication | Authors | Title |
| Journal of | Leveillee, Raymond J., | Radiofrequency Interstitial |
| Endourology, | and Hoey, Michael F., | Tissue Ablation: Wet Electrode |
| Volume 17, | | |
| Number 8, | | |
| October 2003 | | |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY

The present invention is directed to a device and method for transurethral needle ablation of prostate tissue to alleviate BPH using bipolar ablation needles and a virtual electrode. To create the virtual electrode, a conductive fluid is delivered to the target site within the prostate tissue. Ablation energy is then delivered to the tissue and the virtual electrode via a pair of adjacent bipolar ablation needles that penetrate the prostate tissue. The ablation energy flows between the bipolar ablation needles, through the virtual electrode and the prostate tissue to create ablation lesions within the prostate tissue.

Various embodiments of the present invention provide solutions to one or more problems existing in the prior art with respect to the ablation of prostate tissue. The problems include, for example, the fact that a typical lesion created with a “dry” electrode will normally not exceed one centimeter in diameter. This small size stems from several factors. With a dry electrode, the resistive heating which creates the lesion occurs only at or near the needle/tissue interface. Also, the tissue surrounding the needle electrode tends to dessicate as the temperature of the tissue increases. Tissue dessication leads to the creation of a high resistance/impedance to the future passage of current from the needle electrode into the tissue. Once a certain level of impedance is reached, the ablation procedure must sometimes be discontinued because the high impedance limits the size of the lesion that can be created. In addition, to avoid dessication of tissue, ablation energy must be applied slowly, prolonging the procedure. In order to achieve lesions of sufficient size, multiple needle insertions and multiple current applications may be required. Typically, the needles must be retracted, repositioned and redeployed several times during an TUNA procedure, prolonging the procedure, patient recovery time and increasing the potential risks to the patient.

Various embodiments of the present invention solve at least one of the foregoing problems. For example, the present invention overcomes at least some of the disadvantages of the foregoing procedures by providing a device and method capable of achieving larger lesion sizes. Larger lesion sizes can be achieved by performing transurethral ablation using bipolar, virtual electrodes. A transurethral ablation procedure and device, in accordance with the invention, utilizes multiple needles in a bipolar configuration for the ablation of prostate tissue. The invention also provides a transurethral ablation procedure and device utilizing virtual, otherwise referred to as “wet” electrodes. In particular, a fluid is introduced between the bipolar electrodes to provide a bipolar, virtual electrode that covers a larger volume of prostate tissue, resulting in larger lesions. The invention provides improved impedance control and allows for higher levels of RF energy to be delivered to the prostate tissue. Larger lesions can thus be created in a shorter period of time. The number of times that the needles must be repositioned and redeployed is also reduced. All of these factors result in a transurethral ablation device and procedure which is faster and more efficient for the physician to perform. In addition, the invention provides a transurethral ablation procedure which minimizes damage to the urethra and thereby reduces the associated patient pain and longer recovery times.

Various embodiments of the invention may possess one or more features to solve the aforementioned problems in the existing art. For example, the invention provides a transurethral ablation device and method comprising multiple needles in a bipolar configuration. The invention also provides a transurethral device and method comprising use of virtual, otherwise known as “wet,” electrodes. In one embodiment, a pair of bipolar ablation needles is used to deliver ablation energy to the target prostate tissue. One or both of the needles may include fluid delivery ports for the delivery of fluid to the target tissue site. Delivery of the fluid creates a virtual electrode within the prostate. The ablation energy flows between the bipolar ablation needles, throughout the virtual electrode and the corresponding tissue to create a lesion within the prostate. A virtual electrode can be substantially larger in volume than the needle tip typically used in RF ablation and thus can create a larger lesion than can a dry, needle tip electrode. The creation of a virtual electrode enables the RF current to flow with reduced resistance or impedance throughout a larger volume of tissue, spreading the resistive heating created by the current flow through a larger volume of tissue and thereby creating a larger lesion than could otherwise be created using a dry electrode. In addition, the use of multiple, bipolar electrodes can result in a larger lesion size and eliminates use of a ground pad attached to the patient's body.

The invention also provides a transurethral ablation procedure embodied by a method for use of the ablation device described above. The method involves, for example, inserting a distal end of a catheter into a urethra of a male patient, deploying first and second bipolar ablation needles, delivering a conductive fluid to the tissue, and applying ablation energy via the first and second bipolar ablation needles. In this manner, larger lesions can be created in a shorter period of time, with fewer needle insertions into the prostate tissue.

In comparison to known implementations of transurethral prostate ablation, various embodiments of the present invention may provide one or more advantages. In general, the invention may produce larger lesions in a shorter period of time and at the same time reduce the number of times the ablation needles must be inserted into the prostate tissue. Thus, the invention can result in a less complex, more efficient and more convenient procedure. The invention also can result in a procedure in which the risk of damage to the urethra and the associated patient pain and longer recovery times are minimized, thereby promoting patient safety and procedural efficacy.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features and advantages of the invention will be apparent from the description and drawings, and from the claims.

### BRIEF DESCRIPTION OF DRAWINGS

FIG. 1 is a schematic diagram illustrating a device for transurethral ablation of prostate tissue in accordance with the invention.

FIG. 2 is an enlarged view of the distal end of the device of FIG. 1.

FIG. 3A and FIG. 3B are end and side views, respectively, of the distal end of the device of FIG. 1.

FIGS. 4A and 4B are views of two needle systems equipped to deliver fluid to a target tissue site.

FIG. 5 is a side view of an ablation needle equipped to deliver a fluid to a target tissue site.

FIG. 6 is a side view of an alternative ablation needle equipped to deliver a fluid to a target tissue site.

FIG. 7 is a side view of another alternative ablation needle equipped to deliver a fluid to a target tissue site.

FIG. 8 is a side view of another alternative ablation needle equipped to deliver a fluid to a target tissue site.

FIG. 9 is a side view of another alternative ablation needle equipped to deliver a fluid to a target tissue site.

FIG. 10 is a side view of an ablation catheter incorporating two pairs of bipolar ablation needles for delivery of a fluid to target tissue sites.

FIG. 11 shows an end view of a two needle ablation system and the virtual electrode created by the two needles.

FIG. 12 is a flow diagram illustrating a transurethral ablation procedure in accordance with the invention.

### DETAILED DESCRIPTION

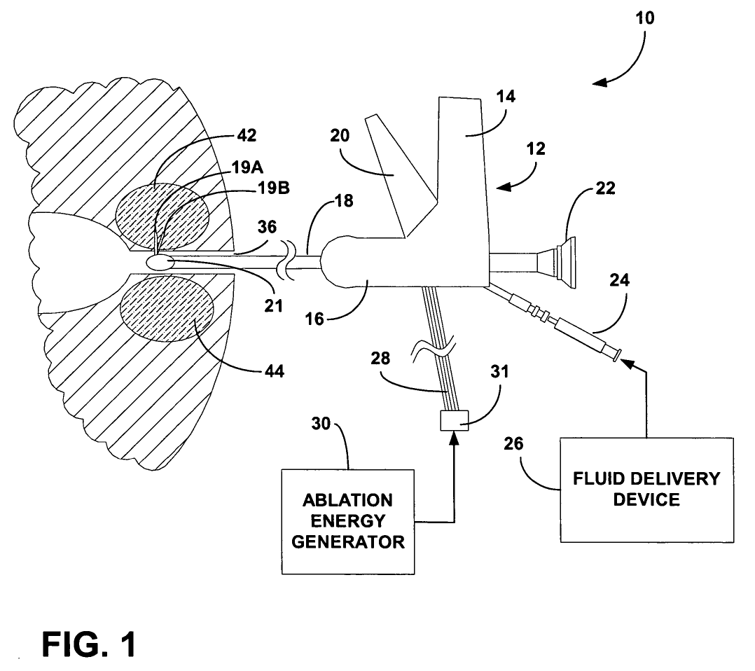

FIG. 1 is a schematic diagram illustrating a device 10 for transurethral ablation of prostate tissue. In accordance with the invention, device 10 includes a pair of bipolar ablation needles and fluid delivery ports for delivery of a fluid to target tissue within the prostate of a patient to create a virtual electrode. The bipolar ablation needles and creation of the virtual electrode allow for more effective and precise ablation. The device may also include other features that will be apparent from this description. Device 10 may generally conform to TUNA devices commercially available from Medtronic, Inc, of Minneapolis, Minn.

As shown in FIG. 1, device 10 includes a manipulator 12 having a handle 14, a barrel 16, and a catheter 18 extending from the barrel. A trigger-like actuator 20 is actuated to advance electrically conductive bipolar ablation needles 19A and 19B from a distal end 21 of catheter 18. Device 10 may further include an endoscope viewfinder 22 coupled to an endoscopic imaging device that extends along the length of catheter 18.

A fluid delivery port 24 is coupled to a fluid delivery lumen (not shown) that extends along the length of catheter 18 to deliver fluid to distal end 21. A proximal end of fluid delivery port 24 is coupled to a fluid delivery device 26 that includes a reservoir containing a fluid and hardware to transmit the fluid to fluid delivery port 24. For example, fluid delivery device 26 may include a pump, a syringe, or other mechanism to transmit the fluid.

An ablation current cable 28 is coupled to an electrical conductor that extends along the length of catheter 18 to needles 19A and 19B. A proximal end of cable 28 is coupled to an ablation energy generator 30 via an electrical connector 31. Ablation energy is applied to the prostate tissue via the bipolar ablation needles 19A and 19B. The needles 19A and 19B are bipolar in the sense that the ablation energy flows between the needles 19A and 19B, through the surrounding prostate tissue to create a lesion. Use of a bipolar needle configuration eliminates the need for a ground pad attached to the patient's skin or other type of return electrode as required by monopolar electrode systems.

In operation, a surgeon introduces catheter 18 into urethra 36 of a male patient, and advances the catheter so that distal end 21 is deployed adjacent the prostate. Endoscopic viewfinder 22 may aid in positioning distal end 21 of catheter 18 relative to the prostate lobes. In particular, distal end 21 is deployed between lateral lobes 42, 44 in the example of FIG. 1. Needles 19 are extended from distal end 21 of catheter 18 to penetrate the urethral wall and one of the prostate lobes 42, 44. In some embodiments, catheter 18 may carry multiple pairs of ablation needles on opposite sides of the catheter to simultaneously access both lobes 42, 44.

Prior to activation of ablation energy generator 30 to deliver ablation current to needles 19, fluid delivery device 26 may be activated to deliver the fluid to the target tissue site proximate prostate 42. For example, fluid delivery device 26 may deliver a fluid that is conductive, such as saline, or a fluid that is loaded with a conductive material. In this manner, the fluid serves the purpose of creating a virtual electrode to enhance the ablation procedure. A virtual electrode can be substantially larger in volume than the needle tip electrode typically used in RF interstitial ablation procedures and thus can create a larger lesion than can a dry, needle tip electrode. That is, the virtual electrode spreads or conducts the RF current density outward from the RF current source into or onto a larger volume of tissue than is possible with instruments that rely on the use of a dry electrode. In other words, the creation of the virtual electrode enables the current to flow with reduced resistance or impedance throughout a larger volume of tissue, thus spreading the resistive heating created by the current flow through a larger volume of tissue and thereby creating a larger lesion than could otherwise be created with a dry electrode.

Either or both of needles 19 or distal end 21 of catheter 21 may include one or more ports for emission of the fluid. The fluid may be sufficiently viscous to provide a controllable flow within catheter 18 and out of distal end 21 of catheter 18. Fluid delivery device 26 may be activated to deliver the fluid before, during and/or after the ablation procedure. For example, the fluid may be delivered before the ablation needles 19A and 19B are activated in order to prepare the tissue in and around prostate gland 42 for delivery of the ablation energy.

Delivery of the fluid prior to ablation establishes the virtual electrode shape and volume. In addition, catheter 18 may continue to deliver the fluid during the course of the ablation procedure to replenish material that may be consumed by the ablation energy. The fluid may also be delivered for a defined period of time after the ablation energy is deactivated and before bipolar needles 19A, 19B are withdrawn from prostate 42. In some embodiments, the concentration of the conductive fluid may be modulated in stages so that different concentrations are delivered within the stages prior to, during and after ablation.

The fluid may be transmitted to the target tissue site, i.e., the region adjacent prostate lobes 42, 44, by a fluid delivery lumen coupled to one or both of needles 19A, 19B. In particular, either one or both of needles 19A or 19B may be hollow and include one or more fluid delivery ports, as will be described below. In another embodiment, either one or both of needles 19A or 19B may include an outer concentric tube defining an annular space for delivery of the fluid. The fluid may also be delivered via fluid delivery tubes associated with one or both of the needles 19A or 19B. Hence, the fluid may be delivered via the same needles 19A or 19B used to deliver ablation energy to prostate lobe 42.

Upon penetration of needles 19A and 19B into prostate lobe 42 and delivery of the fluid to create the virtual electrode, the needles 19A and 19B deliver ablation energy from ablation energy generator 30 to ablate tissue within the prostate lobe. Needles 19A and 19B are bipolar ablation needles wherein ablation current flows between the two needles 19 via the virtual electrode created by the fluid to ablate the prostate tissue.

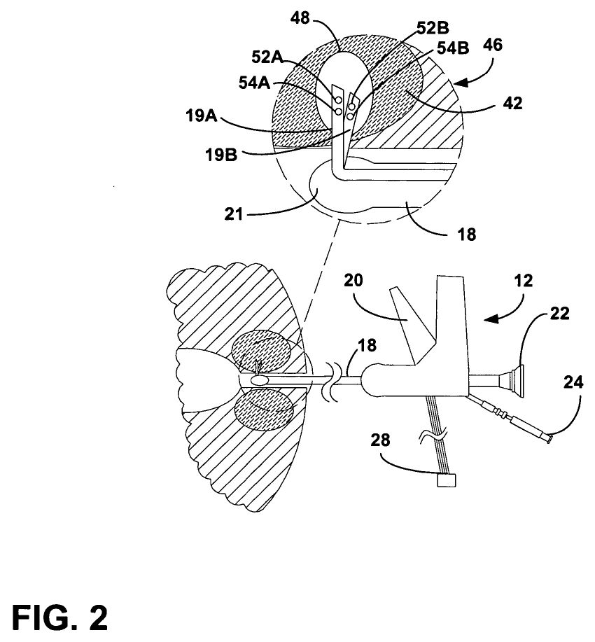

FIG. 2 is an enlarged view of the distal end 21 of device 10 of FIG. 1. As shown in enlarged region 46, distal end 21 of catheter 18 includes an aperture that permits needles 19A and 19B to extend outward from the catheter to penetrate lateral prostate lobe 42. Either one or both of needles 19A and 19B may include fluid delivery ports 52, 54 for delivery of the fluid into the tissue of prostate lobe 42. Upon application of the fluid via one or both of the needles 19A or 19B, the fluid penetrates the tissue interstitially so as to create a virtual electrode 48 within prostate 42. Upon application of ablation current, bipolar ablation needles 19 create a zone of ablated tissue generally defined by virtual electrode 48. Propagation of ablation current and effective ablation of prostate tissue are aided by the conductive fluid dispersed throughout the virtual electrode 48.

Needles 19 may be constructed of a highly flexible, conductive metal such as nickel-titanium alloy, tempered steel, stainless steel, beryllium-copper alloy and the like. Nickel-titanium and similar highly flexible, shaped memory alloys are preferred. Either one or both of needles 19A or 19B may be hollow needles including an internal lumen (not shown in FIG. 2) in fluid communication with fluid delivery ports 52A, 54A, 52B and 54B. Needles 19A and 19B form opposing polarities for bipolar application of RF ablation current. In this manner, current may be generally confined to the region surrounding needles 19A and 19B and the volume of virtual electrode 48.

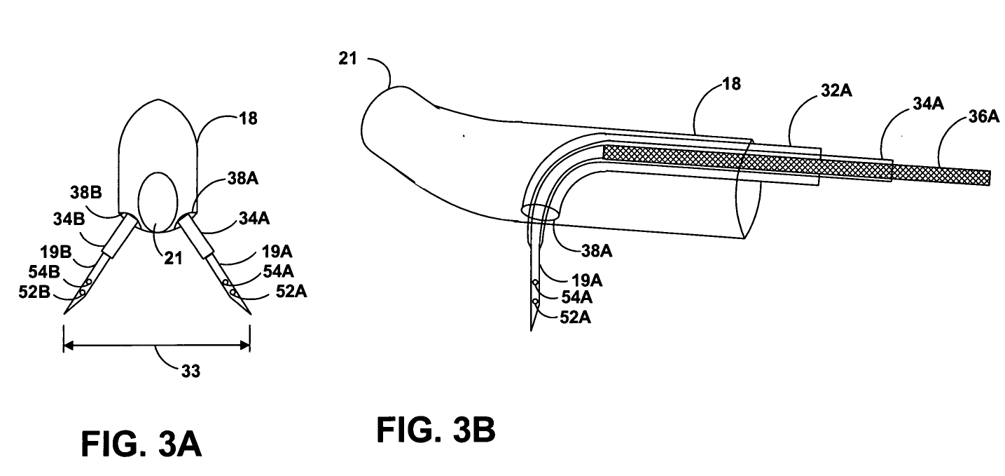

FIG. 3A and FIG. 3B show end and side views, respectively, of the distal end 21 of the device of FIG. 1. An exemplary bipolar, two-needle system is shown in FIGS. 3A and 3B. In this embodiment, catheter 18 includes guide tubes 32A and 32B (in FIG. 3B, guide tube 32B cannot be seen because it is behind guide tube 32A in this view) extending from the proximal to near the distal end 21 of catheter 18. Needle exit ports 38A and 38B are formed in the wall of the catheter body 18 by the guide tubes 32A and 32B, respectively. Push rods 36A and 36B are connected at their proximal end to a mechanism for deploying the needles 19A and 19B. For example, the push rods 36A and 36B may be operationally connected to the trigger-like actuator 20 (see FIG. 1) for deploying and retracting the needles 19A and 19B, respectively, into and out of the prostate tissue. Push rod 36A serves to transfer the mechanical motion of the actuator and thus “push” its respective needle 19A out of the exit port 38A of the guide tube 32A and into the prostate tissue. Similarly, push rod 36B serves to transfer the mechanical motion of the actuator and thus “push” its respective needle 19B out of the exit port 38B of the guide tube 32B and into the prostate tissue. The needles 19A and 19B are inserted into the same prostate lobe such that a complete bipolar ablation circuit can be created between the two needles 19A and 19B in a single prostate lobe during the ablation procedure.

Needles 19 may be disposed adjacent one another in a substantially side-by-side relationship as shown in FIG. 3A. In the embodiment of FIG. 3A, needles 19A and 19B exit from the distal end 21 of the catheter 18 at an angle to each other and thus have different insertion points into the prostate tissue, resulting in two different needle “sticks”. An insulative sheath 34 surrounds each needle 19 and its corresponding push rod 36. In the embodiment shown in FIGS. 3A and 3B, each needle 19A and 19B includes fluid delivery ports 52 and 54 for delivery of fluid to the target tissue site. It shall be understood, however, that either one or both of the needles 19 may include fluid delivery ports. Furthermore, it shall be understood that the invention is not limited to the specific type of fluid delivery ports shown in FIGS. 3A and 3B. Additional configurations of fluid delivery ports will be described below.

Once deployed from the distal tip 21 of the catheter 18, the needles 19A and 19B are physically spaced apart by the distance indicated by reference numeral 33. The needles 19A and 19B may be spaced apart such that they create a sufficiently large ablation zone between the needles. At the same time, the needles may be spaced sufficiently close so that they both penetrate the same prostate lobe. In addition, the needles may be spaced sufficiently close so that both of the needles 19A and 19B are located within the virtual electrode resulting from the delivery of fluid to the tissue. Each needle 19A and 19B may have a total length in the range of approximately 12-22 millimeters, which may be adjustable by the surgeon or which may be fixed in some embodiments. The distance 33 will depend in part upon the length of the needles and the angle between them. In one embodiment, for example, the distance 33 is in the range of 1±0.5 centimeters.

The two needle electrode arrangement described herein has several advantages over other bipolar needle electrode arrangements known in the art. For example, because the ablation needles are spaced apart upon deployment in the prostate, a larger zone of tissue to be ablated is created between the two needles. This is as compared to other bipolar electrode arrangements on a single needle, such as a needle tip/ring electrode arrangement or a coaxial conductor electrode arrangement. This results in a larger area between the source and return electrodes over which the ablation energy travels and thus a correspondingly larger area of tissue ablation. In addition, since both needles may be used to deliver fluid to the target tissue, a larger virtual electrode may be created than when fluid is delivered via a single needle. This may further tend to result in a larger area of tissue ablated. Use of two needles for fluid delivery may also compensate for those times when one of the needles is unable to deliver fluid because of blockages in the fluid delivery ports or conduits, failure of an associated fluid delivery device, or other reason. In that case, the other needle may continue to deliver fluid, creating and sustaining a virtual electrode such that bipolar, virtual electrode needle ablation may continue.

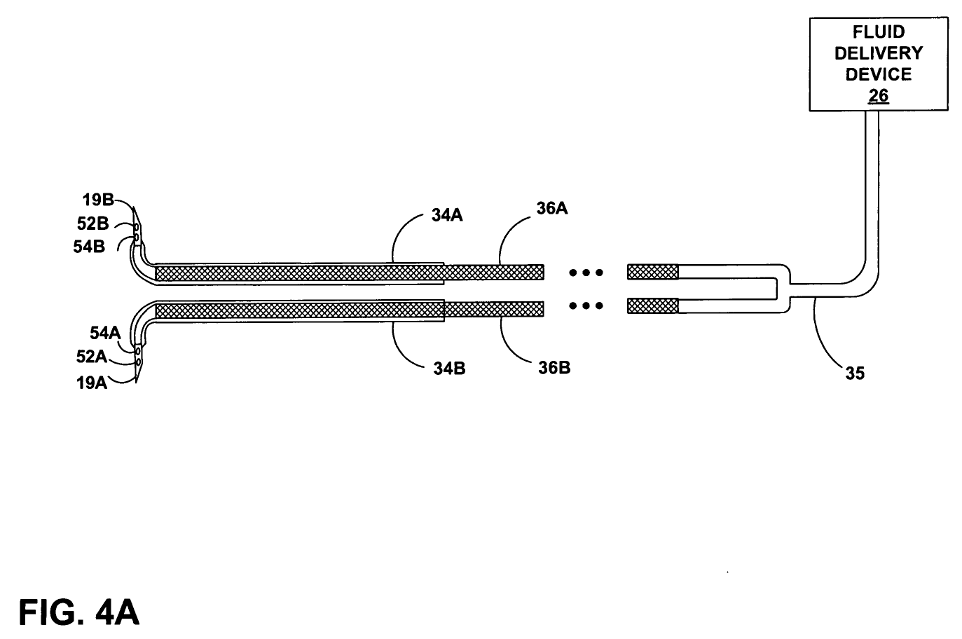

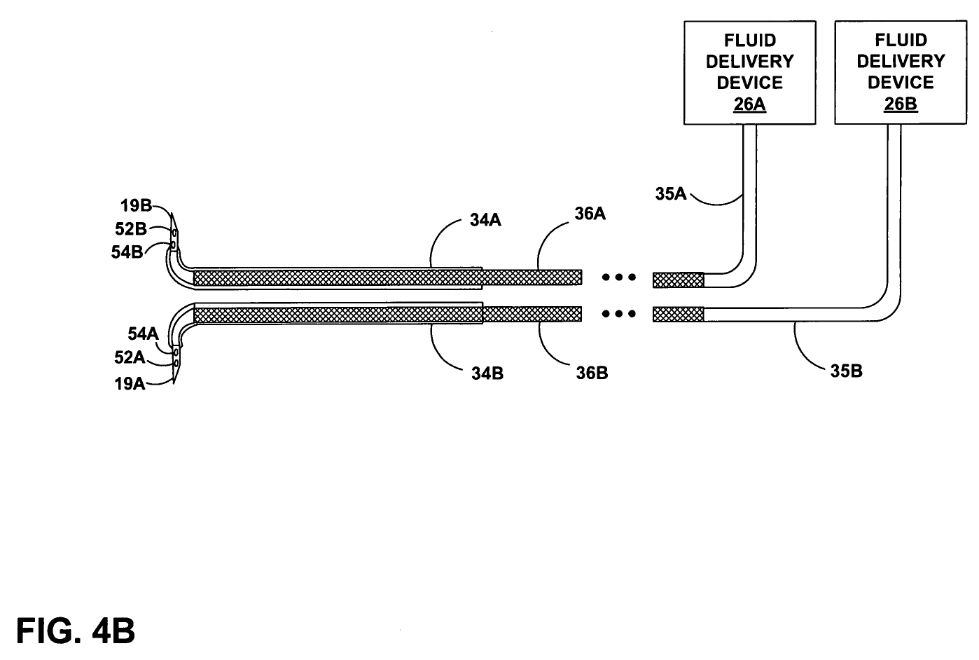

FIGS. 4A and 4B show top, perspective views of two configurations for delivering fluid to the needles 19A and 19B. In the embodiment shown in FIG. 4A, push rods 36A and 36B and needles 19A and 19B are hollow and include fluid delivery ports 52A, 54A and 52B, 54B, respectively, for delivery of the fluid to the target tissue site. Push rods 36A and 36B are connected to receive fluid from fluid delivery device 26 via fluid delivery tube 35. In this sense, push rods 36A and 36B serve as fluid delivery conduits for delivering the fluid from the fluid delivery device to the needles 19A and 19B, respectively. In the embodiment shown in FIG. 4A, fluid delivery tube 35 may be bifurcated to simultaneously deliver fluid to both push rods 36A and 36B, and hence to both needles 19A and 19B. In the embodiment shown in FIG. 4B, two fluid delivery devices 26A and 26B independently deliver fluid via dedicated fluid delivery tubes 35A and 35B, respectively, to their associated push rods 36A and 36B. In this embodiment, the fluid flow rate may be independently controllable for each of the ablation needles 19A and 19B.

Although the embodiments shown in FIGS. 4A and 4B show hollow needles and push rods for the delivery of fluid, it shall be understood that alternative methods of delivering fluid to the target tissue site may be used without departing from the scope of the present invention. Alternate embodiments of ablation devices equipped for fluid delivery will be shown and described in more detail below.

The system described herein is a two-needle, bipolar ablation system. The system is bipolar in the sense that the electrical ablation energy, namely an ablation current, flows between the two electrically conductive, bipolar ablation needles. A bipolar system simplifies the system set up by removing the need for the ground pad required by monopolar ablation systems. Moreover, RF energy is more localized to the prostate. The RF ablation energy is therefore applied only to the precise location of the prostate requiring treatment and therefore lower energy levels can be used and the risk of ablating and/or burning other tissues is greatly reduced.

In general, the electrical ablation current delivered by needles 19A and 19B may be selected to provide pulsed or sinusoidal waveforms, cutting waves, or blended waveforms that are effective in producing the resistive/ohmic/thermal heating which kills cells within the target tissue site. In addition, the electrical current may include ablation current followed by current sufficient to cauterize blood vessels. The electrical current is accompanied by delivery of the fluid, which may be a conductive fluid such as saline or may be a fluid loaded with conductive particles to yield desired conduction characteristics.

The characteristics of the electrical ablation current are selected to achieve significant cell destruction within the target tissue site. The electrical ablation current may comprise radio frequency (RF) current in the range of approximately 5 to 300 watts, and more preferably 5 to 50 watts, and can be applied for a duration of approximately 15 seconds to 3 minutes. If electrocautery is also provided via needles 19, then ablation energy generator 30 also may generate electrocautery waveforms.

In one embodiment, electrical ablation current flows between bipolar ablation needles 19A and 19B. For example, in the two-needle configuration shown in FIG. 2, electrical ablation current may flow between a source needle electrode 19A and the return needle electrode 19B.

Once the needle has been placed in the tissue, pre-ablation infusion of the conductive fluid may begin. The infusion of the conductive fluid creates an interstitial virtual electrode 48. Once the desired level of pre-ablation infusion has occurred, in other words, once the desired virtual electrode size has been approximately achieved, electrical ablation current may be applied to the tissue through the ablation needles 19A and 19B. The needles 19A and 19B serve as source and return conductive electrodes as well as providing conductive fluid delivery ports, although fluid may, but need not be, delivered via both needles 19A and 19B. The virtual electrode 48 may have a substantially spherical, oval or amorphous shape. However, the exact configuration of the virtual electrode will depend upon factors such as tissue irregularities, channels between cells, length of the needles, distance between needle tips, the precise layout of the fluid delivery ports and resulting direction of fluid flow from the needles 19, or any differential fluid flow in a particular direction, among other factors. It shall be understood that the precise shape taken by the virtual electrode is therefore not a limiting factor for purposes of the present invention. The conductive fluid will facilitate the spread of the current density substantially equally throughout the extent of the flow of the conductive fluid, thus creating a virtual electrode substantially equal in extent to the size of the delivered conductive fluid. RF current can then be passed through the virtual electrode into the tissue.

A virtual electrode can be substantially larger in volume/surface area than the needle tip electrode typically used in RF interstitial ablation procedures and thus can create a larger lesion than can a dry, needle tip electrode. That is, the virtual electrode spreads or conducts the RF current density outward from the RF current source into or onto a larger volume/surface area of tissue than is possible with instruments that rely on the use of a dry electrode. In other words, the creation of the virtual electrode enables the current to flow with reduced resistance or impedance throughout a larger volume/surface area of tissue, thus spreading the resistive heating created by the current flow through a larger volume/surface area of tissue and thereby creating a larger lesion than could otherwise be created with a dry electrode. This also allows greater power to be applied while still maintaining a lower current density throughout the virtual electrode.

The fluid can be supplied to the tissue either before the application of ablation energy, at the same time as at least part of the application of ablation energy, throughout the application of ablation energy, or after the application of ablation energy. In one embodiment, the fluid is supplied both pre-ablation and throughout the application of ablation energy.

The ablation energy generator controls the infusion of the fluid into the tissue to be ablated. The ablation energy generator controls the pre-ablation infusion of fluid, infusion of fluid during the ablation procedure itself, and any post-ablation infusion of fluid. The period of pre-ablation infusion and/or the infusion rate can be determined by the user or, alternatively, can be pre-programmed into the ablation energy generator. Similarly, the infusion rate during the ablation procedure may also be determined by the user, or alternatively, can be pre-programmed into the ablation energy generator. In another embodiment, the device may present several possible pre-programmed infusion levels to the user. The user may then choose which levels of infusion are most appropriate based on the particular ablation device to be used, type of needle or needles, type of fluid delivery ports, the type of fluid and the particular patient. In addition, the rate of infusion during the pre-ablation may be the same or may be different than the rate of infusion during the ablation procedure. For example, in a closed loop system, where the impedance and/or the temperature are monitored, the rate of infusion may be varied to control the impedance or the temperature during the ablation process.

To create the virtual electrode, the fluid is delivered to the tissue at a measured rate for a predetermined period of time. In one embodiment, the virtual electrode is created before ablation energy is applied. In another embodiment, delivery of fluid and application of ablation energy begin at substantially the same time. When the virtual electrode is created before application of ablation energy, pre-ablation infusion of fluid occurs for a period of time and at a rate sufficient to create a virtual electrode of the desired size and conductivity. In an embodiment where both needles in a pair of bipolar needles are configured to deliver fluid, the pre-ablation infusion time may be between 5 and 20 seconds at a rate of 0.5-2.0 cubic centimeters (cc)/minute per needle. More particularly, the pre-ablation infusion time may be between 10 and 15 seconds. In one embodiment, the delivery of fluid continues through the application of ablation energy at this same rate unless adjusted by the ablation energy generator in response to, for example, temperature or impedance measurements. The fluid may have a tendency to be vaporized during ablation and therefore fluid may be continuously delivered during the ablation to maintain size and continuity of the virtual electrode. The total length of time that fluid is delivered may be anywhere from 30 seconds to 3 minutes, which may depend in part upon the power applied and the desired lesion size. The total volume of fluid delivered may be anywhere from between 0.5 cubic centimeters to 8 cubic centimeters, which may depend in part upon the rate of fluid flow and the total length of time that the fluid is delivered. The power applied by the ablation energy generator for bipolar needle ablation with the virtual electrode established as described above may be in the range of 15-40 Watts. More particularly, the power applied by the ablation energy generator may be in the range of 20-30 Watts, or 23-27 Watts. The impedance of the target tissue may be maintained anywhere between 10-100 ohms. It shall be understood that the invention is not limited to specific values for the fluid flow rate, volume of fluid delivered, length of fluid delivery time, power applied, tissue impedance or temperature, or any other specific parameter. The values listed above may be examples of possible values for each of these parameters but the invention is not limited in this respect.

As discussed above, the ablation procedure is controlled by the ablation energy generator. To create, maintain and control the virtual electrode, and to control the ablation of the target tissue, at least one of several parameters may be monitored. The applied power and/or the fluid flow may be adjusted in response to these measured parameters. For example, control of the virtual electrode and the ablation procedure may be accomplished in response to measured temperatures of the target tissue and/or measured impedances of the target tissue over predetermined time intervals. Examples of such mechanisms to control the virtual electrode and the ablation procedure are described in U.S. Pat. No. 6,409,722 to Hoey et al. and in U.S. Pat. No. 6,537,272 to Christopherson et al., which are both incorporated herein by reference in their respective entireties.

In some embodiments, the system may first create a virtual electrode in all of the target tissue sites to be ablated, and then return to those sites to deliver the ablation energy. Alternatively, with each needle penetration, or “stick,” the system can inject enough fluid to create a virtual electrode and then ablate before removing the needle. Also, the fluid may be delivered at an efficacious flow rate before, during and after the ablation. Additional effects of constant perfusion with the fluid are natural cooling of the needle tip, which can reduce charring and burning at the needle tip, and potentially result in larger lesions or faster lesions.

The fluid may include a variety of liquids, gels, or liquid suspension containing a variety of conductive materials. For example, the fluid may take the form of a conductive fluid such as isotonic or hypertonic saline. The fluid may also take the form of a biocompatible hydrogel loaded with conductive materials, such as any of a variety of biocompatible, conductive salts, or anesthetic agents. Examples of conductive fluids which may be used include, but are not limited to, NaCl (sodium chloride), CaCl₂ (calcium chloride), MgCl₃ (magnesium chloride), KCl (Potassium chloride), Na₂SO₃ (sodium sulfate), CaSO₄ (calcium sulfate), MgSO₄ (magnesium sulfate), Na₂HPO₄ (sodium hydrogen phosphate), Mg₃(PO₄)₂ (magnesium phosphate tribasic), NaHCO₃ (sodium bicarbonate), CaCO₃ (calcium carbonate) or MgCO₃ (magnesium carbonate). “Ringer's” solution, an isotonic, aqueous solution of the chlorides of sodium, potassium, and calcium, could also be used. The conductive fluid serves to conduct RF electrical current throughout the volume of the fluid applied to the prostate, thereby increasing the effective volume of the lesion created by application of ablation current.

In addition to being conductive, the fluid may also be loaded with an anesthetic agent, an antiseptic, or an anti-inflammatory. As an example of a suitable anesthetic agent, a gel material loaded with approximately 18 to 20 ml of 1% lidocaine, will achieve a desired anesthetic effect when applied to the prostate tissue. Examples of anesthetic agents includes benzocaine, dyclonine, markaine, sensorcaine, lidocaine, and lidocaine hydrochloride gel, or mixtures thereof. Other possible anesthetic agents are Benzocaine, Butamben, Tetracaine, Dibucaine, Dyclonine, Lidocaine, and Pramoxine or mixtures thereof. In some embodiments, it may be desirable to include a vasoconstrictor to keep the anesthetic effect localized. The prostate is highly vascularized and highly innervated. The highly innervated prostate and relatively localized area of delivery may limit the anesthetic effect. With excellent vascularization, is very likely for anesthetic transference across the prostate via the highly vascularized perfusion system of the prostate. The vaso-constrictor tends to reduce blood flow that otherwise would contribute to cooling in the ablation zone, and thereby reduce the concentration of ablation energy and prolong the time needed for effective ablation.

The fluid delivered via the transurethral ablation catheter 18 may also include a steroid to promote healing of prostate tissue following the ablation procedure. The steroid may be mixed with the conductive fluid. The steroid may be delivered before, during or after the ablation procedure. Alternatively, the steroid may be delivered independently of the conductive/anesthetic fluid. For example, the steroid may be delivered following the ablation procedure to promote the healing of the prostate tissue.

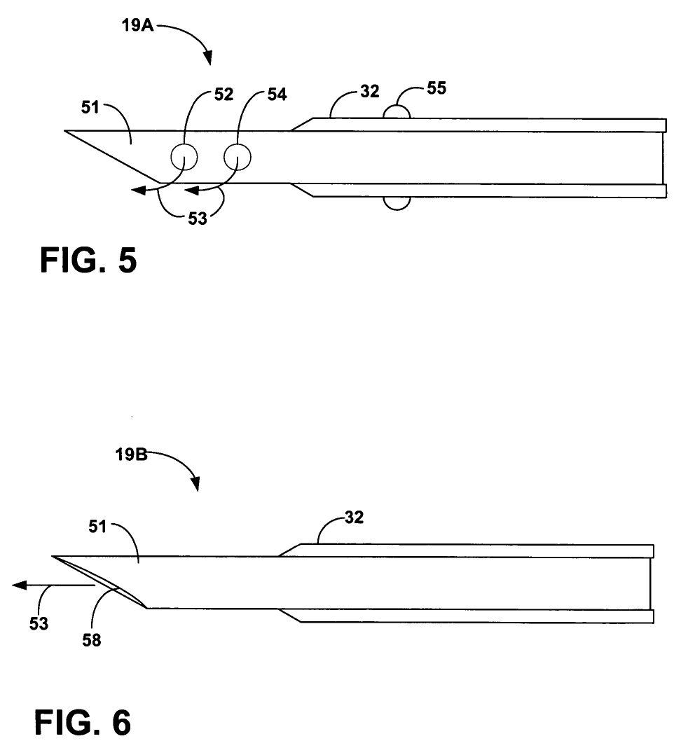

FIG. 5 is a side view of one of the ablation needles 19A equipped to deliver a fluid to a target tissue site. It shall be understood that, in each of FIGS 5-10, either one or both needles in the pair of bipolar needles 19 may be configured for fluid delivery. For simplicity of illustration, however, only one needle is shown in each of FIGS. 5-10.

As shown in FIG. 5, ablation needle 19A may include an insulative sheath 56 and a needle body 51. In this embodiment, needle body 51 is hollow and includes an interior lumen or passage (not shown) for delivery of fluid. The fluid can be pumped through the lumen to one or more fluid delivery ports 52, 54 through which the fluid may flow into the tissue to be ablated. Fluid flow is indicated generally in each of FIGS. 5-10 by reference numeral 53. The number of fluid delivery ports 52, 54 may vary. In addition, additional fluid delivery ports may be formed at opposite sides of needle body 51, or at different circumferential positions about the periphery of the needle body 51. The embodiment of FIG. 5 also shows an annular ring 55 circumferentially disposed about insulative sheath 56. Ring 55 serves to block the space where the needle penetrates the urethral wall and to thus prevent the flow of fluid back into the urethra. It shall be understood that ring 55 may also be present on any of the other embodiments shown and described herein.

The length of needle 19 may be on the order of approximately 12 to 22 mm. However, needle lengths of up to 50 mm may be desirable to deliver the fluid to the ends of the prostatic capsule. Additionally, it may be desirable to perfuse the fluid through some or all of the entire 50 mm depth to create a virtual electrode, and then withdraw the needle to the 12 to 22 mm needle depth range to perform the ablation.

FIG. 6 is a side view of another ablation needle 19B equipped to deliver a fluid to a target tissue site. In the example of FIG. 5, needle 19B includes a distal fluid delivery port 58 at the distal tip of needle body 51 through which fluid may be delivered to the tissue to be ablated as indicated by arrow 53.

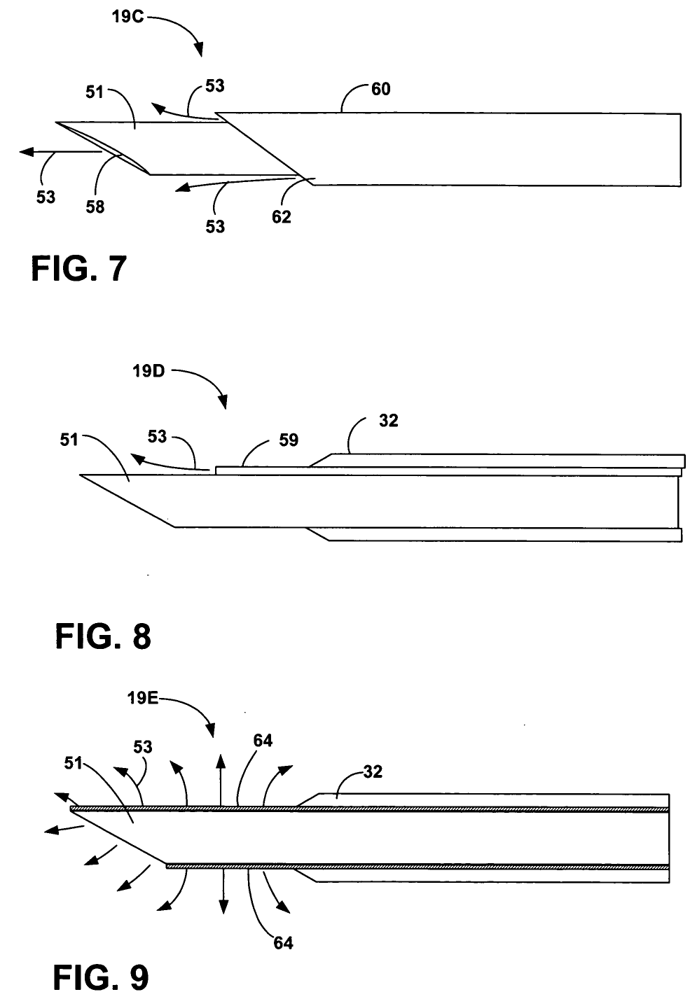

FIG. 7 is a side view of another alternative ablation needle 19C equipped to deliver a fluid to a target tissue site. In the example of FIG. 7, ablation needle 19C includes a concentric tube arrangement comprising needle body 51 and an outer tube 60. The annular space defined between outer tube 60 and needle body 51 forms a fluid delivery port 62. The outer tube 60 may be positioned between the insulative sheath 32 and the needle body 51 as shown in FIG. 7, or it may be positioned outside of the insulative sheath 32. In some embodiments, needle body 51 also may include a distal fluid delivery port 58 such as that shown in FIG. 6.

FIG. 8 is a side view of another alternative ablation needle 19D equipped to deliver a fluid to a target tissue site. Ablation needle 19D includes a fluid delivery tube 59 through which fluid is delivered to the prostate tissue. The fluid delivery tube 60 may be positioned between the insulative sheath 32 and the needle body 51 as shown in FIG. 8, or it may be positioned outside of the insulative sheath 32.

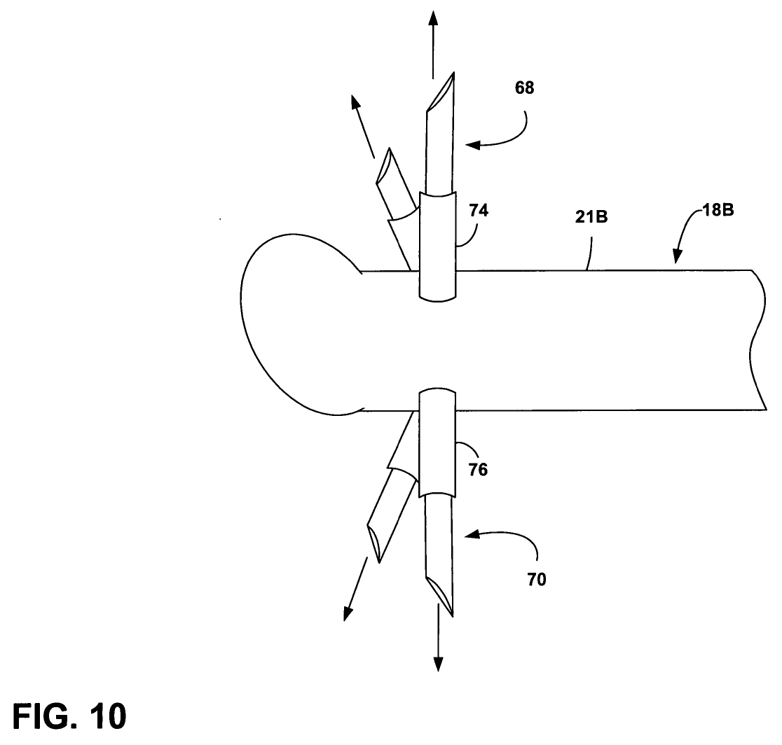

FIG. 9 is a side view of another alternative ablation needle 19E equipped to deliver a fluid to a target tissue site. In this embodiment, the needle body 51 is coated with a porous surface 64 through which the conductive fluid exudes into the surrounding tissue in a substantially uniform manner as indicated by reference numerals 53. The needle body 51 may also include fluid delivery ports (not shown) through which the fluid is delivered to the porous surface. The porous surface 64 itself may not be conductive, but electrical conduction may occur via the conductive fluid in the porous material. Examples of the porous material may include any of a number of microporous, non-conductive materials such as silicone, polytetrafluoroethylene (PTFE), expanded polytetrafluoroethylene (EPTFE), polyurethane, polyester, dacron fabric, biocompatible hydrogel, cintered polyethylene material or cintered metals. The pores in the material can be large enough to allow the conductive fluid to flow freely, but not too large where they would become clogged with tissue.

FIG. 10 is a side view of a distal end 21B of an ablation catheter 18B incorporating two pairs of bipolar ablation needles 68, 70 for delivery of ablation current and a fluid. Pairs of ablation needles 68, 70, each forming a bipolar electrode set, may be mounted at positions appropriate for access to two of the prostate lobes, such as the right lateral and left lateral lobes. Each of the needles 68, 70 may extend from respective insulative sheaths 74, 76. In the example shown in FIG. 10, pairs of bipolar ablation needles 68, 70 define respective distal fluid delivery ports for delivery of the fluid such as that shown in FIG. 6. However, it shall be understood that any of the embodiments shown in FIGS. 5-9 may be used for the delivery of fluid and that the invention is not limited in this respect. In one embodiment, the pairs of bipolar ablation needles 68, 70 may be deployed and retracted simultaneously to reach their respective target tissue sites. In another embodiment, the pairs of bipolar ablation needles 68, 70 may be independently deployable to provide greater flexibility to the surgeon during the ablation procedure.

In operation, using manipulator 12 (see FIGS. 1 and 2), the surgeon may initially translate and rotate catheter 18, for example, to bring needles 19 into alignment with one of the prostate lobes. If catheter 18 includes only a single pair of bipolar needles, the surgeon may rotate the catheter, following ablation of tissue within the first target lobe, to access the other lateral lobe and the medial lobe, if desired. Alternatively, as mentioned above with respect to FIG. 10, catheter 18 may include two or more pairs of bipolar needles oriented to penetrate two lobes simultaneously. Longitudinal and radial positioning of catheter 18 may be aided by endoscopic viewfinder 22, or other imaging techniques such as ultrasound, MRI or the like.

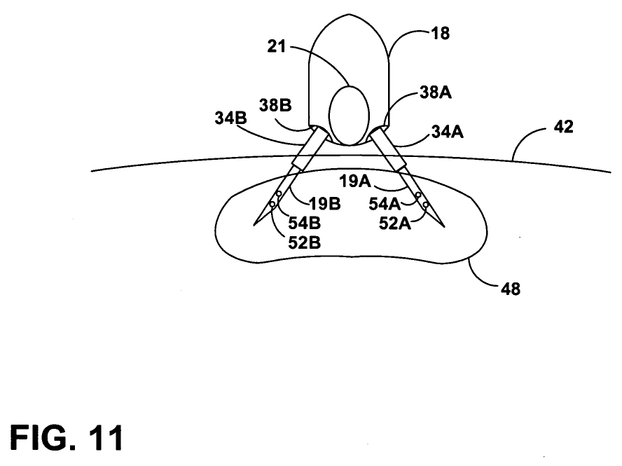

FIG. 11 shows an end view of a two needle ablation catheter 18 and a virtual electrode 48. Upon deployment of distal end 21 proximate a target tissue site within the urethra, ablation needles 19A and 19B are inserted into the prostate tissue 42. For example, a surgeon may use actuator 20 (FIG. 1) to drive needles 19A and 19B through the urethral wall and into prostate tissue 42. Needles 19A and 19B may be inserted together by a single action of the surgeon or they may be separately controlled. When needles 19A and 19B are lodged in the prostate tissue 42, the surgeon activates fluid delivery device 26 (FIG. 1) to deliver the fluid along the length of catheter 18, through the fluid delivery conduits and the push rods to needles 19A and 19B. Needles 19A and 19B deliver the fluid to the prostate tissue to create a volume of conductive fluid for use as a virtual electrode 48.

After creation of the virtual electrode 48, the surgeon activates ablation energy generator 19 to deliver ablation energy to the tissue site via needles 19A and 19B. The ablation current flows between the two bipolar needles 19A and 19B and throughout the virtual electrode and ablates a zone of tissue. The tissue ablated may correspond generally to the volume/surface area of the virtual electrode 48. If desired, the surgeon may continue to deliver the fluid to the target tissue site during the delivery of ablation current. Fluid may also be delivered following the ablation procedure before withdrawing needle 19A and 19B from the target tissue site.

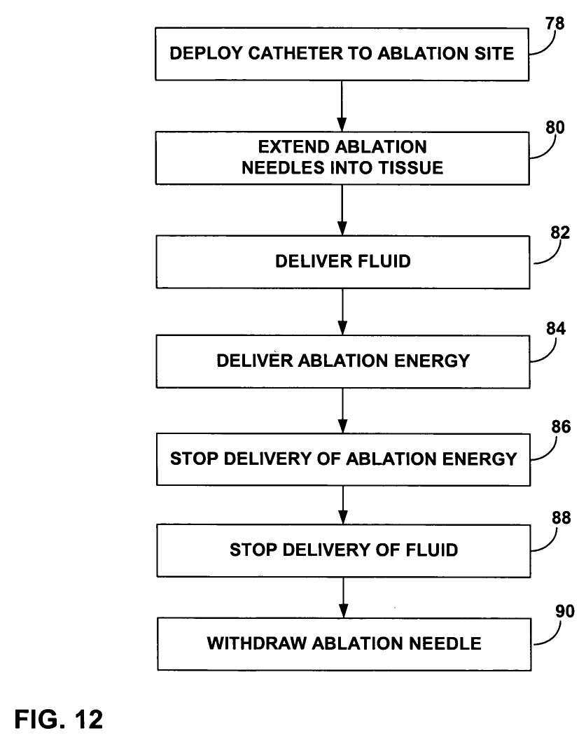

FIG. 12 is a flow diagram illustrating a transurethral ablation procedure. The procedure involves deploying a catheter to an ablation site (78). For example, deploying a catheter transurethrally to a position within the urethra corresponding to the target prostate tissue to be ablated. Upon extension of the ablation needles into the target tissue (80), fluid is delivered (82) to the target tissue site within the prostate to create a virtual electrode. The fluid may be delivered continuously during the ablation procedure to maintain the virtual electrode.

Once the virtual electrode is created, ablation energy is applied (84). The ablation energy ablates cells within the target tissue site. When delivery of the ablation energy is stopped (86), delivery of the fluid may also be stopped (88). Alternatively, the fluid may continue to be delivered for a period of time following termination of the ablation energy, particularly if an anesthetic or steroid is to be delivered post-ablation. Then, the ablation needle and catheter may be withdrawn from the patient (90).

It shall be understood that somewhat different procedures may be followed without departing from the scope of the present invention. For example, in other embodiments, pre-ablation delivery of fluid may not occur and instead fluid delivery and application of ablation energy may be initiated at substantially the same time.

As further features, a controller may be provided to coordinate the timing and duration of delivery of ablation current and the fluid by ablation energy generator 30 and fluid delivery device 26, respectively. For example, the controller may execute a surgeon-programmable routine to selectively activate fluid delivery during the course of ablation.

The invention can provide a number of advantages. In general, the invention provides greater volumetric coverage and precision in the ablation procedure, enabling a greater volume of prostate tissue to be more uniformly ablated within a given ablation procedure. The invention provides improved impedance control and allows for higher levels or RF energy to be delivered to the prostate tissue. Larger lesions can thus be created in a shorter period of time. Because the lesions produced may be larger, the number of times that the needles must be repositioned and redeployed is also reduced. The use of a bipolar needles and virtual electrodes shortens overall ablation time and reduces the number of needle “sticks”, thus minimizing damage to the urethra and the associated patient pain and longer recovery times. All of these factors result in a transurethral ablation device and procedure which is faster and more efficient for the physician to perform. In addition, in some embodiments, the fluid can be delivered by the same device used to perform the transurethral ablation procedure, making the procedure less complex, quicker, and more convenient for the surgeon.

As a further advantage, the virtual electrode formed by fluid delivery supports controlled ablation within a larger, yet more precise, zone of prostate tissue. With continued delivery of fluid during ablation, the efficacy of the lesion either in size, or time to develop lesion size, may be improved. In addition, continued delivery of fluid during ablation may reduce or eliminate the need for fluid delivery to cool the urethra, e.g., by delivering fluid out of the catheter and into the urethra.

As a further advantage, in those embodiments where an anesthetic agent is used, the invention may reduce the pain associated with some existing transurethral ablation techniques. Also, the invention offers a localized treatment for alleviation of pain. This embodiment of the invention also eliminates the need for a transperineal prostatic block, sedation or general anesthesia. The most common block is the perineal prostatic block which typically is done under ultrasound guidance. The invention removes the need to have an ultrasound device to deliver pain medication, and removes the need for additional equipment, e.g., syringe and needle, to deliver the perineal prostatic block. In this manner, the invention simplifies delivery of pain relief along with ablation delivery.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for transurethral ablation, as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A method for performing transurethral needle ablation, comprising:
deploying first and second ablation needles to a target tissue site within a prostate of a male patient, wherein the first and second ablation needles form a bipolar electrode pair;
delivering an electrically conductive fluid to the target tissue site; and
delivering ablation energy to the target tissue via the bipolar ablation needles.

2. The method of claim 1, further comprising delivering the fluid via fluid delivery ports carried by at least one of the ablation needles.

3. The method of claim 1, further comprising delivering the fluid via a fluid delivery port at a distal tip of at least one of the ablation needles.

4. The method of claim 1, further comprising delivering the fluid via an annular space formed by at least one ablation needle and an outer tube concentric with the at least one ablation needle.

5. The method of claim 1, further comprising delivering the fluid via a fluid delivery tube.

6. The method of claim 1, further comprising delivering the fluid before the delivery of the ablation energy.

7. The method of claim 1, further comprising delivering the fluid during the delivery of the ablation energy.

8. The method of claim 1, further comprising stopping delivery of the ablation energy, and delivering the fluid after stopping delivery of the ablation energy.

9. The method of claim 1, wherein the ablation energy includes electrical current selected to kill cells within the prostate.

10. The method of claim 1, wherein the fluid includes at least one of an anesthetic agent, an antiseptic, or an anti-inflammatory.

11. The method of claim 1, wherein delivering the electrically conductive fluid includes creating a virtual electrode within the prostate.

12. The method of claim 1, wherein delivering ablation energy comprises delivering an radio frequency ablation current that flows between the bipolar ablation needles.

13. The method of claim 1, further comprising penetrating a wall of a urethra of the patient with the bipolar ablation needles, extending the ablation needles into the prostate, delivering the fluid to the prostate via at least one of the ablation needles, and delivering the ablation energy to the prostate via the ablation needles.

14. The method of claim 1, wherein the fluid comprises saline.

15. The method of claim 14, wherein the saline comprises one of isotonic and hypertonic saline.

16. The method of claim 1, wherein the fluid is a gel having conductive particles suspended therein.

17. The method of claim 1, wherein the fluid includes one of NaCl (sodium chloride), CaCl₂ (calcium chloride), MgCl₃ (magnesium chloride), KCl (Potassium chloride), Na₂SO₃ (sodium sulfate), CaSO₄ (calcium sulfate), MgSO₄ (magnesium sulfate), Na₂HPO₄ (sodium hydrogen phosphate), Mg₃(PO₄)₂ (magnesium phosphate tribasic), NaHCO₃ (sodium bicarbonate), CaCO₃ (calcium carbonate), MgCO₃ (magnesium carbonate) or Ringer's solution.

18. The method of claim 1 wherein deploying a set of bipolar ablation needles further includes deploying the bipolar ablation needles such that a distance between needle tips is approximately 1±0.5 centimeters.

19. A transurethral ablation system comprising:
a transurethral catheter;
a first ablation needle extendable from the catheter to penetrate a prostate of a patient;
a second ablation needle extendable from the catheter to penetrate a prostate of a patient, wherein the first and second ablation needles form a bipolar electrode pair;
a fluid delivery conduit extending within the catheter;
a fluid delivery device to deliver fluid to the prostate via the fluid delivery conduit; and
an ablation energy generator to deliver ablation energy to the prostate via the first and second ablation needles and the fluid.

20. The system of claim 19, further comprising a fluid delivery port formed in at least one of the bipolar ablation needles and coupled to the fluid delivery conduit.

21. The system of claim 19, wherein the fluid delivery device delivers an electrically conductive fluid to the prostate.

22. The system of claim 21, wherein delivery of the electrically conductive fluid creates a virtual electrode within the prostate.

23. The system of claim 19, wherein the ablation energy and the fluid are both delivered to the prostate via the first and second ablation needles.

24. The system of claim 19, wherein the fluid delivery conduit includes a fluid delivery port in at least one of the bipolar ablation needles.

25. The system of claim 19, wherein the fluid delivery conduit includes fluid delivery ports in both of the bipolar ablation needles.

26. The system of claim 25, wherein the fluid delivery conduit is connected to supply the fluid to both bipolar ablation needles.

27. The system of claim 25, wherein the fluid delivery conduit includes first and second fluid delivery conduits, each of the conduits corresponding to one of the first and second bipolar ablation needles, and wherein each of the first and second fluid delivery conduits are connected to supply the fluid to its respective one of the first and second bipolar ablation needles.

28. The system of claim 27, wherein the fluid delivery device includes first and second fluid delivery devices connected to deliver fluid to the first and second fluid delivery conduits, respectively.

29. The system of claim 19, wherein the ablation energy is a current that flows between the first and second ablation needles.

30. The system of claim 19, further including fluid delivery ports carried by at least one of the ablation needles.

31. The system of claim 19, further including a fluid delivery port at the distal tip of at least one of the ablation needles.

32. The system of claim 19, further including an annular space for delivering the fluid formed by at least one of the ablation needles and an outer tube concentric with the at least one ablation needle.

33. The system of claim 19, further including a fluid delivery tube.

34. The system of claim 19, wherein the fluid includes saline.

35. The system of claim 34, wherein the fluid includes at least one of isotonic and hypertonic saline.

36. The system of claim 19, wherein the fluid includes at least one of an anesthetic, an antiseptic, an anti-inflammatory or a vaso-constrictor.

37. The system of claim 19, wherein the fluid includes one of NaCl (sodium chloride), CaCl₂ (calcium chloride), MgCl₃ (magnesium chloride), KCl (Potassium chloride), Na₂SO₃ (sodium sulfate), CaSO₄ (calcium sulfate), MgSO₄ (magnesium sulfate), Na₂HPO₄ (sodium hydrogen phosphate), Mg₃(PO₄)₂ (magnesium phosphate tribasic), NaHCO₃ (sodium bicarbonate), CaCO₃ (calcium carbonate), MgCO₃ (magnesium carbonate) or Ringer's solution.

38. The system of claim 19 wherein the fluid delivery device delivers fluid to the first and to the second ablation needles.

39. The system of claim 19 wherein a distance between needles tips of the first and second ablation needles is approximately 1±0.5 centimeters.

40. The system of claim 19 further including:
a first fluid delivery conduit associated with the first ablation needle;
a second fluid delivery conduit associated with the second ablation needle;
a first fluid delivery device to deliver fluid to the prostate via the first fluid delivery conduit and the first ablation needle; and
a second fluid delivery device to deliver fluid to the prostate via the second fluid delivery conduit and the second ablation needle.

41. The system of claim 19 further including first and second annular rings, each of the annular rings corresponding to and circumferentially disposed about one of the first and second bipolar ablation needles.

42. A transurethral ablation system, comprising:
means for creating a virtual electrode at a target tissue site within the prostate of a male patient; and
means for delivering ablation energy between a first needle electrode and a second needle electrode and the electrically conductive fluid.

43. The system of claim 42 wherein the means for creating a virtual electrode comprises means for delivering a conductive fluid to the target tissue site.

44. The system of claim 43 wherein the means for delivering a conductive fluid comprises fluid delivery ports located in at least one of the first or second needle electrodes.

45. The system of claim 44 wherein the means for delivering a conductive fluid further comprises at least one fluid delivery device.
